(19) United States
(12) Patent Application Publication
(10) Pub. No.: US 2018/0303484 A1
(43) Pub. Date: Oct. 25, 2018

(54) NEGATIVE-PRESSURE TENSION-RELIEF SUTURE DEVICE FOR SKIN WOUND

(71) Applicant: SICHUAN RUI ZE JIAN TECHNOLOGY CO. LTD., Chengdu, Sichuan (CN)

(72) Inventor: Xinping Zhang, Nanchong (CN)

(73) Assignee: Xiaoping Du, San Jose, CA (US)

(21) Appl. No.: 15/769,326

(22) PCT Filed: Oct. 18, 2016
(86) PCT No.: PCT/CN2016/102384

§ 371 Date: Apr. 18, 2018

(30) Foreign Application Priority Data
Oct. 19, 2015 (CN) 201510680819.1

(51) Int. Cl. A61B 17/08; A61M 1/00

(52) U.S. Cl. CPC A61B 17/085; A61B 17/04; A61M 1/0023; A61M 1/0088

(57) ABSTRACT

A negative-pressure tension-relief suture device for a skin wound, comprises: a negative-pressure support (1); a sealing membrane (2); and deformable members (3). The deformable members (3) are disposed at two sides of the negative-pressure support (1). An air void is formed on the negative-pressure support (1). The sealing membrane (2) covers the deformable members (3) to form a closed space. The negative-pressure support (1) communicates an interior of the closed space with the outside. The negative-pressure tension-relief suture device for a skin wound discharges tissue fluid from a skin wound while relieving tension, thereby reducing negative influence of tension on the skin wound, and automatically causing alignment of edges of the skin wound.

The present application is the U.S. national phase of PCT Application No. PCT/CN2016/102384 filed on Oct. 18, 2016, which claims a priority of the Chinese patent application No. 201510680819.1 filed on Oct. 19, 2015 and entitled “NEGATIVE-PRESSURE TENSION-RELIEF SUTURE DEVICE FOR SKIN WOUND”, which is incorporated herein by reference in its entirety.

### TECHNICAL FIELD

The Invention relates to a negative-pressure tension-relief suturing device for skin wounds, especially a negative-pressure tension-relief suturing device for skin wounds in the medical treatment field.

### BACKGROUND

The modern closure plan for surgical incisions or wounds must simultaneously consider relevant factors affecting wound healing such as: timely cleaning and drainage of exudate and sphacelus of wounds, elimination of dead spaces of skin incisions, flattening and alignment of skin edges of wounds, tension reduction of wounds, prevention of new wounds caused by suturing, and protection for moist coverage of wounds.

In prior art, after the wound is covered with negative-pressure dressing, it is only possible to clean and drain exudate and sphacelus, while requirements for alignment and flattening of skin edges of incisions and tension reduction of wounds (i.e. tension reduction at two sides of the skin wound) still remain unsatisfied. With the closing device for skin incisions in prior art, it is only possible to simply pull the two sides of the skin incision towards the centerline. In the pulling-together process, the device needs to rely on external forces and complicated pulling-together structures and steps. Moreover, stress on the skin is uneven; particularly, the irregular wounds with curved shapes will lead to some parts of the wounds excessively pulled while other parts unaligned. Besides, the device cannot clean and drain the exudate and sphacelus of skin incisions, nor eliminate dead spaces of skin incisions, nor satisfy requirements for suturing of irregular wounds with curved shapes, and nor protect wounds with moist coverage. No device in prior art considers about providing a wound support structure to ensure alignment and flattening of skin edges of wounds. Therefore, in prior art, there is no complete closure plan for skin wounds, i.e., a negative-pressure tension-relief suturing device that can timely clean and drain the exudate and sphacelus of wounds, eliminate dead spaces of incisions, ensure close alignment and flattening of skin edges of wounds, reduce tension evenly and effectively, avoid new wounds caused by suturing, and protect wounds with moist coverage.

### SUMMARY

The technical problems to be solved by the present invention are to provide a negative-pressure tension-relief suturing device for skin wounds which can conduct negative-pressure cleaning and drainage of sphacelus of wounds, eliminate dead spaces of skin incisions, pull skin edges of wounds to align automatically, assist for alignment and flattening of skin edges of wounds, adapt to suturing for irregular wounds with curved shapes, and ensure moist coverage of wounds. Meanwhile, it also provides comfortable use and easy maintenance.

The negative-pressure tension-relief suturing device for skin wounds in the Invention for coping with the above technical problems comprises a negative-pressure support, a sealing membrane and deformable members; the deformable members are arranged at two sides of the negative-pressure support; an air hole is arranged on the negative-pressure support; the sealing membrane covers the deformable members to create a confined space; the inside and the outside of the confined space created by the sealing membrane are made communicated by the negative-pressure support.

Furthermore, the device also comprises a horizontal end face; the horizontal end face is arranged at the lower part of the negative-pressure support.

Furthermore, the negative-pressure support has an ellipse-like section, and an intersecting end plane is arranged at the lower part of the ellipse-like section; the intersecting end plane of the ellipse-like section is correspondingly parallel to the skin wound.

Furthermore, the deformable members are placed at two sides of the wound; bonding layers are arranged on the skin-contacting surfaces of the deformable members, and the skin-contacting surfaces of the deformable members are fixedly connected to the skin surface of the wound through the bonding layers.

Furthermore, the deformable members are arranged with transitional zones at one side beyond the negative-pressure support.

Furthermore, the deformable members are of a split-type structure created by splicing multiple tension-relief segments together.

Furthermore, a pressure gauge is connected to the negative-pressure support.

The beneficial effects of the Invention are: by making advantage of negative pressure, the device is able to make the deformable members deform relative to the skin surface of the wound, the sealing membrane and the surface of the negative-pressure support, thus producing a tightly fitted structure. Since the negative-pressure support has an elliptical section and its horizontal end face is correspondingly parallel to the skin wound (6), after a negative-pressure environment is created, it will automatically pull the skin edges at both sides of the skin wound to move towards the center of the wound and the intersecting end plane and fit tightly, and automatically align and flatten the skin edges with the help of the horizontal end face of the corresponding negative-pressure support. Wherein, when the deformable members drive the skin wound attached to it to contract to the center, a tension-relief force is then produced to offset tension of the wound, thus realizing the tension-relief suturing function for the wound; with the help of the horizontal end face of the corresponding negative-pressure support, wound alignment and flattening is also realized; under negative pressure and through the air hole on the negative-pressure support, the sphacelus of the wound can be cleaned and drained under negative pressure, and dead spaces of the skin incision can be eliminated forcibly; multiple tension-relief segments of deformable members can be spliced together to adapt to the suturing for irregular wound with curved shapes; the coverage of the sealing membrane to the negative-pressure support, the deformable members, the skin and the skin wound can provide a healing environment with moist; the integrated negative-pressure tension-relief suturing device can be easily operated, is a combination of multiple functions which are necessary for wound healing, and can realize accurate alignment and flattening of skin edges of the wound, reliable tension reduction, effective elimination of dead spaces and adequate cleaning and drainage of sphacelus of the wound. The device is able to ensure a growth environment with moist coverage for the wound. In the Invention, the deformable members deform under internal negative pressure without help of any external structure, which makes the device smaller and easier for use and maintenance. Besides, the deformable members are surrounded by negative pressure, which makes the deformation more sufficient, so as to improve the tension-relief effect of the skin wound.

### BRIEF DESCRIPTION OF THE DRAWINGS

FIG. 1 is a front view of the device of the present invention, in accordance with an embodiment.

FIG. 2 is a sectional view of the device of the present invention, in accordance with an embodiment.

Main parts and locations with corresponding numbers in the drawings are as follows: negative-pressure support 1, sealing membrane 2, deformable members 3, bonding layers 4, transitional zones 5, skin wound 6, and intersecting end plane 7.

### DETAILED DESCRIPTION OF THE PREFERRED EMBODIMENTS

This invention is further described in combination with drawings as follows:

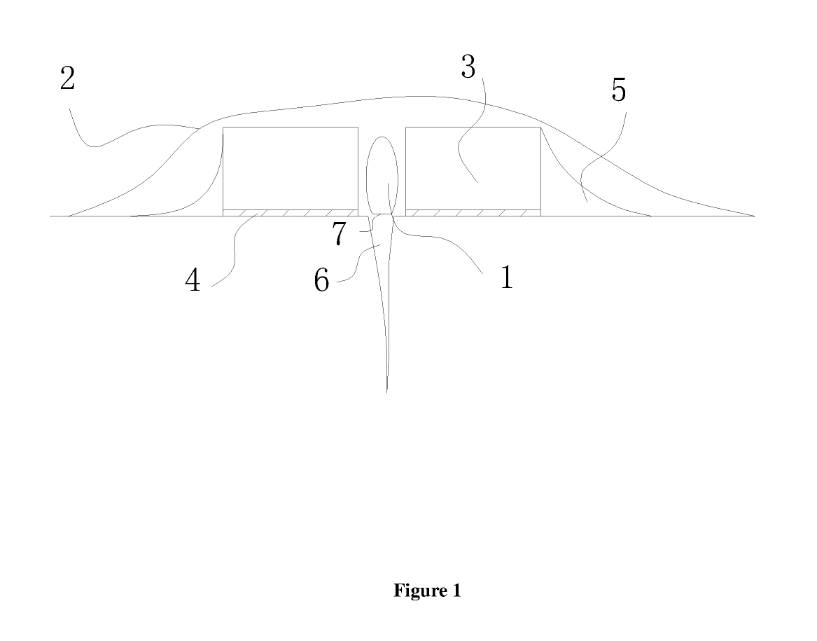

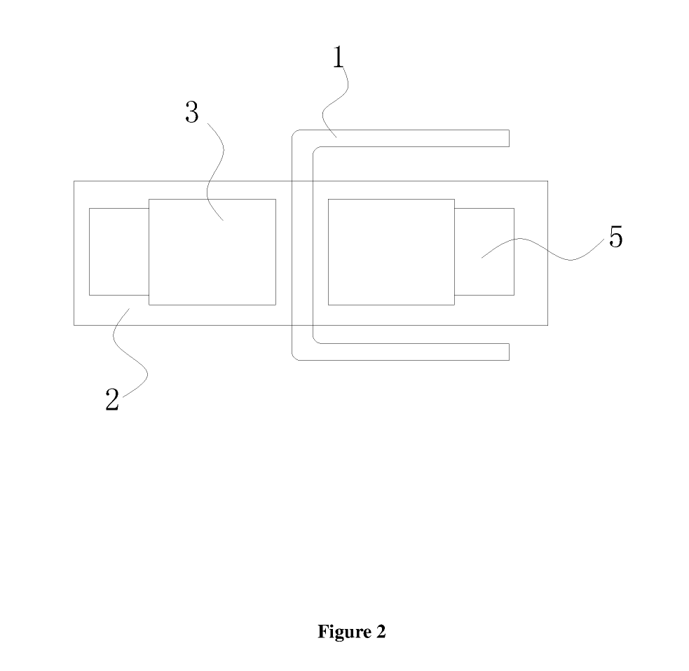

As shown in FIGS. 1 and 2, the negative-pressure tension-relief suturing device for skin wounds in an embodiment of the present invention for coping with the above technical problems comprises a negative-pressure support 1, a sealing membrane 2 and deformable members 3; the deformable members 3 are arranged at two sides of the negative-pressure support 1; an air hole is arranged on the negative-pressure support 1; a horizontal end face is arranged at the lower part of the negative-pressure support 1; the sealing membrane 2 covers the deformable members 3 to create a confined space; the inside and the outside of the confined space created by the sealing membrane 2 are made communicated by the negative-pressure support 1. The deformable members 3 can be made of elastomers, plastomers and other materials which can deform under pressure. The sealing membrane covers the negative-pressure support 1, deformable members 3, the skin and the skin wound, creating a confined space between the deformable members 3, between the negative-pressure support 1 and the sealing membrane, and among the deformable members, the negative-pressure support, the skin and the skin wound and the sealing membrane.

After a negative-pressure environment is created, the above confined space narrows and pulls the skin edges at two sides of the skin wound to move toward the center, and automatically align and flatten the skin edges with the help of the horizontal end face of the corresponding negative-pressure support 1. In service, the device is covered on the skin wound 6, wherein the deformable members 3 are placed at two sides of the skin wound 6 and are pressed closely against the skin, while the negative-pressure support 1 is placed over the skin wound 6. The sealing membrane 2 should be properly covered to ensure a confined space within the membrane, then the negative-pressure support 1 is used to make the confined space become a negative-pressure space.

Under negative pressure, the deformable members 3 move towards the negative-pressure support 1 and fit tightly with it, and the excessive space in the sealing membrane 2 is removed. In the process, the deformable members 3 deform and pull the skin attached to the deformable members 3. Because the negative pressure can make the deformable members 3 fully fitted to the negative-pressure support and the skin at two sides of the wound evenly pressed, under the pulling of the deformable members 3, the skin can align sufficiently and leave no gap. In the process of making a negative-pressure space, the exudate of the wound is drawn out of the sealing membrane 2 through the air hole on the negative-pressure support 1 under negative pressure, so as to clean and drain the exudate and sphacelus of the skin incision, eliminate dead spaces of the skin incision, ensure hygiene of the skin wound 6, and reduce the risk of repeated infection. The coverage of the sealing membrane to the negative-pressure support 1, the deformable members 3, the skin and the skin wound can provide a healing environment with moist.

The device can also comprise a horizontal end face; the horizontal end face is arranged at the lower part of the negative-pressure support 1, according to an embodiment. It can make the deformable members 3 fit more easily with the negative-pressure support under negative pressure and align them under limitation of the horizontal end face, thus making the healed skin wound more regular. Wherein, the horizontal end face can be designed as an integrated structure with the negative-pressure support, i.e., a horizontal face can be directly manufactured at the lower part of the negative-pressure support. Alternatively, the horizontal end face can be made into a component and positioned by being connected to the deformable members 3 or the negative-pressure support.

The negative-pressure support 1 has an elliptical section, and an intersecting end plane 7 is arranged at the lower part of the elliptical section. The intersecting end plane of the elliptical section is correspondingly parallel to the skin wound 6. The above mentioned elliptical section is produced by arranging a horizontal end face, i.e. the intersecting end plane, at the lower part of the elliptical section. By using the elliptical section, the deformable members 3 can fit more easily with the negative-pressure support 1 under negative pressure. In actual application, the sum of the distances between the unattached sections of the skin edges at both sides of the wound should be slightly greater than the width of the intersecting end plane of the negative-pressure support.

Bonding layers 4 are arranged on the deformable members 3, according to an embodiment. The bonding layers are arranged on the skin-contacting surfaces of the deformable members and the skin-contacting surfaces of the deformable members are fixedly connected to the skin surface of the wound through the bonding layers. One side of the bonding layer is fixedly connected to the deformable member and the other side is tightly attached to the skin. The bonding layers can be made by medical glue. Wherein the bonding layers can also be made by blending or mixing medical glue into the deformable members to make the skin-contacting surfaces of the deformable members have enough viscosity and to make the skin-contacting surfaces of the deformable members 3 fixedly connected to the skin surface of the wound.

A pressure gauge is connected to the negative-pressure support 1. The pressure gauge is used to indicate the negative pressure value of the space inside the sealing membrane 2. The tension-relief force can be precisely controlled by the negative pressure value.

The deformable members 3 are arranged with transitional zones at one side beyond the negative-pressure support 1. In order to facilitate tight fitting of the sealing membrane 2 with the components inside after making a negative-pressure space and to remove excessive space, the transitional zones are arranged at one side of the deformable members 3. The transitional zones can be designed as an integrated structure with the deformable members 3, or as a split-type structure.

The deformable members 3 can be of a split-type structure created by splicing multiple tension-relief segments together. In order to adapt to the tension-relief requirement for curved wounds, the deformable members 3 can be divided into multiple tension-relief segments, which can be spliced together at certain angles along the curve of the wound to create each of the deformable members 3. Under negative pressure, all opposite tension-relief segments and adjacent tension-relief segments at two sides of the wound deform along the curved wound and the surface of the negative-pressure support, removing the gap between them to make them fitted with each other. In this way, even the wound has a curve shape, the deformable members 3 can also fully adapt to the irregular and curved shape of the wound after a negative-pressure environment is created, and can ensure adequate alignment and flattening of skin edges of the wound between the tension-relief segments.

According to an embodiment of the present invention, the negative-pressure support is of a hollow pipe structure arranged along the upper surface of the wound, and its bottom corresponds to the upper surface of the skin wound 6. Its bottom section is a horizontal parallel surface, i.e. the intersecting end plane 7. The intersecting end plane can provide a datum support plane for the tightly fitted skin edges of the wound and make the skin edges of the wound align and flatten more easily on the datum plane. Preferably, an ellipse-like shape is adopted for the upper section. Compared with a rectangular or round or other shape of pipeline, a pipeline with ellipse section is able to allow more space for deformation of the skin-bonding surfaces of the deformable members 3 and the sealing membrane-contacting surface, and is favorable to pull skin edges of the wound to align towards the centerline, make the deformed deformable members 3 fit tightly with the negative-pressure support of the wound, and remove the excessive space within the sealing membrane 2.

What is claimed is:

1. A negative-pressure tension-relief suturing device for skin wounds, characterized by comprising a negative-pressure support (1), a sealing membrane (2) and deformable members (3); the deformable members (3) are arranged at two sides of the negative-pressure support (1); an air hole is arranged on the negative-pressure support (1); the sealing membrane (2) covers the deformable members (3) to create a confined space; the inside and the outside of the confined space created by the sealing membrane (2) are made communicated by the negative-pressure support (1).

2. The negative-pressure tension-relief suturing device for skin wounds according to claim 1, characterized by further comprising a horizontal end face; the horizontal end face is arranged at the lower part of the negative-pressure support (1).

3. The negative-pressure tension-relief suturing device for skin wounds according to claim 1, characterized in that: the negative-pressure support (1) has an ellipse-like section, and an intersecting end plane (7) is arranged at the lower part of the ellipse-like section; the intersecting end plane (7) of the ellipse-like section is correspondingly parallel to the skin wound (6).

4. The negative-pressure tension-relief suturing device for skin wounds according to claim 1, characterized in that: bonding layers (4) are arranged on the deformable members (3).

5. The negative-pressure tension-relief suturing device for skin wounds according to claim 1, characterized in that: the deformable members (3) are arranged with transitional zones (5) at one side beyond the negative-pressure support (1).

6. The negative-pressure tension-relief suturing device for skin wounds according to claim 5, characterized in that: the deformable members (3) are of a split-type structure created by splicing multiple tension-relief segments together.

7. The negative-pressure tension-relief suturing device for skin wounds according to claim 1, characterized in that: a pressure gauge is connected to the negative-pressure support (1).
